(19) United States
(12) Patent Application Publication
(10) Pub. No.: US 2026/0256359 A1
(43) Pub. Date: Sep. 3, 2026

(54) NON-INVASIVE SENSOR AND MEASUREMENT METHOD

(71) Applicant: ECLYPIA, Grenoble (FR)

(72) Inventors: Cyrielle MONPEURT, Grenoble (FR); Romain BLANC, Grenoble (FR); Alexandre GALLEGOS, Paris (FR)

(21) Appl. No.: 18/862,911

(22) PCT Filed: May 11, 2023
(86) PCT No.: PCT/EP2023/062675

§ 371 Date: Nov. 4, 2024

(30) Foreign Application Priority Data
May 12, 2022 (EP) 22173132.6

(51) Int. Cl. A61B 5/00; A61B 5/145

(52) U.S. Cl. CPC A61B 5/0095; A61B 5/14532; A61B 5/7278

(57) ABSTRACT

The invention relates to a method for measuring a parameter in a stratified target environment (2) using a non-invasive sensor (1) based on photoacoustic or photothermal detection. The method includes: a) providing a sensor with a light source (11a), a device to control multiple irradiation parameters, a detection cell (12), a memory storing a nodal frequency abacus, and an adaption module (14); b) the adaption module (14) selects an initial model configuration of the stratified target environment (CMirrad) for irradiation; c) the adaption module's processor (14) determines in the nodal frequency abacus a plurality of nodal modulation frequencies and selects at least one optimal modulation frequency (foptim); d) the light source (11a) irradiates the stratified target environment according to this case; e) the detection cell (12) detects the acoustic or thermal signal generated; f) the adaption module's processor (14) determines the parameter of interest based on the detected signal.

### FIELD OF THE INVENTION

The present invention relates to a process measurement and a non-invasive sensor making it possible to measure one or more parameters of interest in a target environment.

More precisely, the invention relates to a non-invasive sensor based on the detection of a photothermal effect or photoacoustic, in particular configured to measure one or more parameters in a target stratified environment, the stratification of which may evolve over time. A measured parameter can for example be blood sugar in the skin.

### TECHNOLOGY BACKGROUND

In the field of sensors for living organisms, it is known to realize non-invasive sensors based on photoacoustic or photothermal detection.

A zone of interest of an environment to be analyzed, called target, is irradiated by means of a laser beam of chosen wavelength and modulation frequency depending on the parameter of interest to be measured. The laser beam is absorbed by the target over a characteristic length which depends on the structuring of the target. Energy absorption light causes local heating of the target. In reaction to this heating, a thermal wave of frequency equal to the laser modulation frequency is generated in the target. This wave propagates in the target and in particular up to the exterior surface of the target.

The thermal wave can be directly detected and analyzed. We then speak of photothermy. Photoacoustics detection exploits the fact that the thermal wave is associated with a pressure wave frequency identical to the modulation frequency.

In the case of photoacoustic detection indirect, we detect the pressure wave generated in the fluid external environment when the thermal wave generated in the target reaches, after propagation, the interface target—fluid external environment.

The photoacoustic effect has been the subject of numerous theoretical studies. Allan Rosencwaig and Allen Gersho have notably developed a theoretical model of the photoacoustic signal. This model involves the physico-chemical properties of the sample to be analyzed, including the optical diffusion length, the thickness and the thermal diffusion length of the sample. (Rosencwaig, A. and Gersho, A. (1976), Theory of the photoacoustic effect with solids, Journal of Applied Physics, 47, 64).

Hu et al. have developed a theory generalized photoacoustic effect in a stratified material. (Hu, H., Wang, X., & Xu, X. (1999). Generalized theory of the photoacoustic effect in a multilayer material. Journal of Applied Physics, 86, 3953-3958.)

Photoacoustic detection presents many advantages compared to other techniques of detection among which we can cite the orthogonal aspect of transduction: the optical signal at the input of the environment to be analyzed is converted into an acoustic signal which is very specific to the phenomenon to be observed and which allows the use of inexpensive and miniaturized sensors.

The difficulty of photoacoustic detection or photothermal comes among others:
the number of parameters generally influencing the detected signal and,
for certain analytes of interest present in concentration weak in the environment to be analyzed, of the weak proportion of the detected signal specific to each of these parameters of interest.

Photoacoustic or photothermal detection requires not only a choice of wavelength, but also a choice of laser modulation frequency(ies). Indeed, the characteristic penetration length of the incident light wave into the target depends on the laser modulation frequency. If the structure of the target (e.g. the skin) changes over time, the modulation frequency(ies) to be used to measure the parameter of interest (e.g. interstitial blood sugar) with controlled accuracy and power consumption will also change over time.

Moreover, in a target stratified environment, reflections at the interfaces between the various strata give rise to a multitude of so-called “secondary” and “tertiary” waves, from a so-called “primary” thermal wave propagating towards the surface of the target environment, which are likely to interfere with one another, as many authors have shown. The interference of thermal waves generated by the photothermal effect has been described in the following documents:
C. A. Bennett and R. R. Patty, “Thermal wave interferometry: a potential application of the photoacoustic effect,”
Appl. Opt. 21, 49-54 (1982);
Andreas Mandelis, “Theory of photothermal-wave diffraction and interference in condensed media,” J. Opt. Soc. Am. A 6, 298-308 (1989);
Andreas Mandelis and Kwan F. Leung, “Photothermal-wave diffraction and interference in condensed media: experimental evidence in aluminum,” J. Opt. Soc. Am. A 8, 186-200 (1991).

These interferences are likely to affect the accuracy of a non-invasive sensor based on photoacoustic or photothermal detection. In a target stratified environment whose stratification is likely to evolve, interference conditions are also likely to evolve, since they depend on the structuring of the target environment. Consequently, interference conditions are not constant over time.

The invention therefore aims to improve the accuracy of a non-invasive sensor in a target environment, in particular a stratified and/or evolving one based on photoacoustic or photothermal detection, by taking account of interference phenomena while controlling the energy consumption of this sensor, or to reduce the energy consumption of this sensor while controlling its accuracy.

### SUMMARY OF THE INVENTION

Thus, the invention relates to a method of measurement of a parameter of interest in a target environment means of a non-invasive sensor based on the photoacoustic detection or photothermal detection. The measuring method comprises:
a) a sensor is provided comprising:
   a light source,
   a device for controlling a plurality of irradiation parameters of the light source, the plurality of irradiation parameters comprising at least one frequency for modulating the intensity of the light source,
   a detection cell configured to detect an acoustic or thermal signal (12),
   a memory in which is stored a nodal frequency chart comprising:
   for a plurality of groups of model configurations of the target stratified environment, each comprising at least two model configurations of the target stratified environment differing from one another only in the parameter of interest,
   and for a plurality of irradiation case groups, each comprising at least two irradiation cases differing from one another only in a modulation frequency of a light source, each irradiation case comprising a set of irradiation parameter values, a plurality of nodal multiplets, each nodal multiplet comprising characteristics common to all the elements of a given group of model configurations of the target stratified environment and a plurality of associated nodal modulation frequencies, for which the acoustic or thermal signal detected by the detection cell in response to irradiation by the light source exhibits a correlation below a predetermined threshold with the parameter of interest;
   and an adaptation module comprising a processor and exchanging information with the detection cell and the light source irradiation parameter control device;
b) the adaptation module selects a model configuration of the initial target stratified environment (CMirrad) for irradiation;
c) the adaptation module processor:
   determines in the nodal frequency chart a plurality of nodal modulation frequencies associated with a group of model configurations of the target stratified environment to which the chosen model configuration of the stratified environment belongs,
   then determines at least one particular modulation frequency on the basis of said plurality of nodal modulation frequencies, said particular modulation frequency being a weighted average of at least two of said plurality of nodal modulation frequencies and being by two nodal modulation frequencies,
   and determines a particular irradiation case for the selected model configuration of the target stratified environment, the particular irradiation case comprising the at least one particular modulation frequency;
d) the light source irradiates the target stratified environment according to the set of irradiation parameters of said particular irradiation case;
e) the detection cell detects an acoustic or thermal signal generated in response to irradiation;
f) the adaptation module processor determines the parameter of interest on the basis of the acoustic or thermal signal detected.

Thanks to these provisions, irradiation is carried out according to an irradiation case comprising a modulation frequency of at least one light source, particular in that it has been chosen to take account of the nodal frequencies corresponding to the model configuration of the target stratified environment chosen for irradiation. In particular, the particular modulation frequency can be determined on the basis of two nodal frequencies, so that the particular modulation frequency corresponds to a sensor sensitivity maximum (all other things being equal) for the interval between these two modulation frequencies.

These provisions therefore make it possible to improve the sensitivity of the non-invasive sensor compared with a sensor of the prior art that does not include this step of adapting the irradiation case by taking interference phenomena into account.

Furthermore, it is possible to select a limited number of modulation frequencies for irradiation, for example by retaining only the particular modulation frequency or a small number of modulation frequencies comprising this particular modulation frequency.

Determining the particular modulation frequency as a weighted average of, and bounded by, the nodal frequencies takes into account the fact that the modulation frequency corresponding to the maximum sensitivity with respect to the parameter of interest lies in the interval between two successive nodal modulation frequencies, but that the characteristic penetration depth of the incident light wave decreases with modulation frequency. To improve the accuracy of the non-invasive sensor, it may therefore be advantageous not to choose the arithmetic mean of two nodal modulation frequencies as the particular modulation frequency, but rather a weighted average of these nodal modulation frequencies, in particular by assigning a higher weight to the lowest nodal modulation frequency.

These provisions therefore make it possible to reduce irradiation power while maintaining sensitivity identical to that of a prior art sensor.

According to one embodiment of the measurement method,
the processor of the adaptation module further implements an inverse modeling algorithm receiving as input an irradiation case and an acoustic or thermal signal and providing as output a model configuration of the target stratified environment and a value of the parameter of interest,
and step f) comprises the following steps:
   f1) the adaptation module processor receives as input the acoustic or thermal signal detected by the detection cell and the particular irradiation case used for irradiation, and returns as output from the inverse modeling algorithm a model configuration of the current target stratified environment and an estimated parameter of interest value;
   f2) the adaptation module processor evaluates the chosen target stratified environment model configuration by comparison with the current target stratified environment model configuration, and only if this evaluation is unfavorable: g) the adaptation module receives as input the model configuration of the current target stratified environment and returns as output a new model configuration of the target stratified environment chosen for irradiation, then c), d), e) and f1) are repeated;
   f3) the value of the parameter of interest measured by the sensor is the last value of the parameter of interest estimated.

The evaluation of the model configuration of the target stratified environment CMirrad is considered unfavorable when the model configuration of the target stratified environment CMirrad is different from the model configuration of the target stratified environment CMmes.

Thanks to these provisions, it is possible to obtain a blood sugar measurement with:
either a single irradiation (no execution of substep g, in the case of a favorable evaluation). This irradiation is then carried out according to an irradiation case chosen by default but corresponding to controlled energy consumption (the irradiation parameters being chosen from a limited number, for example). Step f2), which evaluates the model irradiation configuration, ensures that the measurement accuracy is as high as possible for the case in question, given the irradiation cases available in the correspondence table, which is an advantage over a process in which step g) is not provided and for which this certainty is not available;
or two irradiations (step f2) with execution of sub-step g), in the case of an unfavorable evaluation) and therefore comprising a reiteration of c), d), e) and f1)). The second irradiation is then performed according to the irradiation case that achieves the highest possible accuracy for the case in question in view of the additional information acquired from the first irradiation and the irradiation cases available in the correspondence table. The accuracy of the process is therefore known and improved compared to a process without step g). Furthermore, not only does the selected irradiation case take account of interference phenomena for the model configuration chosen for the first irradiation, but also the optimal nature of the model configuration with respect to the current state of the stratified environment is verified, and if necessary another model configuration and another irradiation case taking account of interference phenomena are chosen for a second irradiation on the basis of which the parameter of interest will be determined.

In this way, the accuracy and/or sensitivity and/or energy consumption of the non-invasive sensor can continuously be improved as the target stratified be evolves.

According to one embodiment of the measurement process, g1) further comprises reiterating f2) at the end of f1).

Thanks to this arrangement, the choice of model configuration and irradiation case, taking into account interference phenomena, is reiterated until the desired sensor performance in terms of accuracy and/or sensitivity and/or energy consumption of the non-invasive sensor is achieved. So we don't limit ourselves to one or two irradiations, three or more irradiations can ultimately achieve better sensitivity for the situation of the target stratified environment at the moment of measurement than with one or two irradiations, or a better sensibility/accuracy/energy consumption compromise. Once again, the energy consumption for each irradiation is controlled by the fact that the number of irradiation parameters is limited for each irradiation case, and that these irradiation parameters take account of interference phenomena, even if convergence is only achieved after three, four, five or even ten successive irradiations, overall energy consumption can be controlled and, in particular, can be lower than that which would be required to acquire a complete spectrum in irradiation frequency and/or modulation frequency, while simultaneously controlling and even improving measurement accuracy.

According to one embodiment of the measurement process, the process comprises in advance:
I—by means of a processor and a database of model configurations comprising multiplets (model configuration of the target stratified environment, irradiation case, parameter of interest) and an acoustic or thermal signal detected by the detection cell associated with each of said multiplets, a nodal frequency chart is generated for:
   a plurality of groups of model configurations,
   and a plurality of groups of irradiation cases,
the nodal frequency chart comprises a plurality of nodal multiplets, each nodal multiplet comprising characteristics common to all elements of a group of model configurations of the target stratified environment and a plurality of associated nodal modulation frequencies, and this nodal frequency chart is stored in the memory of the non-invasive sensor.

Thanks to this arrangement, a nodal frequency chart can be generated by simulation, i.e. in silico, in a much shorter time and covering a much more complete range of model configuration groups and irradiation cases than if the chart had been generated manually. This arrangement further enhances sensor accuracy. It also reduces the cost and manufacturing time of the non-invasive sensor.

According to one embodiment, the measurement method comprises:
II—a processor learns at least one inverse modeling algorithm from the configuration database models and storing the at least one inverse modeling algorithm in the memory of the non-invasive sensor.

Thanks to this arrangement, one or more inverse modeling algorithms can be trained in an automated manner, for example each adapted to a given model configuration and irradiation case or to a group of given model configurations and irradiation cases, which makes it possible to gain in accuracy in the inverse problem solving step required to determine a value of the parameter of interest from the photoacoustic or photothermal signal detected signal. According to one embodiment of the measurement process, at least some of the acoustic or thermal signals detected by the detection cell associated with the multiplets stored in the database of model configurations are simulated, i.e. they are generated by means of a computerized simulation device.

Thanks to this arrangement, a large number of irradiation case groups and configuration groups can be generated by simulation, i.e. in silico, in a much shorter time and covering a much more complete range of model configuration and irradiation case groups than is accessible through in vivo experience. This arrangement further enhances sensor accuracy. It also reduces the cost and manufacturing time of the non-invasive sensor.

According to one embodiment of the measurement method, the particular modulation frequency determined in step c) is a weighted average of at least two of said plurality of nodal modulation frequencies.

The invention also relates to a non-invasive sensor based on photoacoustic or photothermal detection configured to measure a parameter of interest in a stratified target environment comprising:
a light source,
a device for controlling the irradiation parameters of the light source,
a detection cell configured to detect an acoustic or thermal signal,
a memory in which is stored a nodal frequency chart comprising, for:
a plurality of groups of model configurations of the target stratified environment, each comprising at least two model configurations of the target stratified environment differing from one another only in the parameter of interest,
and a plurality of groups of irradiation cases, each comprising at least two irradiation cases differing from one another only in a modulation frequency of a light source, each irradiation case comprising a set of irradiation parameter values, a plurality of so called “nodal” multiplets, each multiplets comprising characteristics common to all the elements of a given group of model configurations of the target stratified environment and a plurality of associated nodal modulation frequencies, for which the acoustic or thermal signal detected by the detection cell in response to irradiation by the light source exhibits a correlation below a predetermined threshold with the parameter of interest,
   the non-invasive sensor further comprising an adaptation module comprising a processor and adapted to exchange information with the detection cell and the light source irradiation parameter control device,
   the adaptation module being further configured to:
select a model configuration of the initial target stratified environment,
determine in the nodal frequency chart a plurality of nodal modulation frequencies associated with a group of model configurations to which the chosen model configuration of the target stratified environment belongs,
determine at least one particular modulation frequency on the basis of said plurality of nodal modulation frequencies, said particular modulation frequency being a weighted average of at least two of said plurality of nodal modulation frequencies and being by two nodal modulation frequencies, and
determine a particular irradiation case for the chosen irradiation model configuration comprising the at least one particular modulation frequency;
transmit a particular irradiation case to the light source irradiation parameter control device,
receive an acoustic or thermal signal detected by the detection cell,
determine the value of the parameter of interest on the basis of the acoustic or thermal signal detected.

In one embodiment of the non-invasive sensor, the processor of the adaptation module is further adapted to:
implement an inverse modeling algorithm receiving as input an irradiation case comprising a set of irradiation parameters and an acoustic or thermal signal, and outputting a model configuration of the target stratified environment and a value of the parameter of interest;
determine by means of the inverse modeling algorithm a model configuration of the current target stratified environment and a value of the parameter of interest estimated on the basis of a detected photoacoustic or photothermal signal received and a chosen irradiation case,
evaluate a chosen model irradiation configuration of the target stratified environment by comparison with a model configuration of the current target stratified environment,
only if the irradiation model configuration of the selected target stratified environment and the model configuration of the current target stratified environment compared are different: determine a new model configuration of the selected target stratified environment for irradiation when it receives a model configuration of the current target stratified environment,
determine in the nodal frequency chart a plurality of nodal modulation frequencies associated with a group of model configurations of the target stratified environment to which the chosen new model configuration of the stratified environment belongs,
then determine at least one particular modulation frequency on the basis of said plurality of nodal modulation frequencies, said particular modulation frequency being a weighted average of at least two of said plurality of nodal modulation frequencies and being delimited by two nodal modulation frequencies,
determine a new particular irradiation case for the newly selected model configuration of the target stratified environment, the particular irradiation case comprising the at least one particular modulation frequency,
and transmitting the new particular irradiation case to the light source irradiation parameter control device so that the light source irradiates the target stratified environment according to the set of irradiation parameters of said new particular irradiation case, the detection cell detects an acoustic or thermal signal generated in response to this new irradiation and the processor of the adaptation module determines the parameter of interest on the basis of the detected new acoustic or thermal signal;
determine the value of the parameter of interest measured on the basis of the last value of the parameter of interest estimated.

According to an embodiment of the non-invasive sensor, at least two of the light source, the device for controlling the irradiation parameters of the light source, the detection cell configured to detect an acoustic or thermal signal, the memory in which a nodal frequencies chart is stored and the adaptation module are mechanically independent of each other. This type of implementation enables a distributed sensor to be formed, so that the space requirement or weight is reduced at the level of the target stratified environment. In particular, the simulation module can be deported for remote management, possibly shared between several users. Finally, the invention relates to a computer program comprising instructions which lead the non-invasive sensor according to one of the preceding embodiments to execute the steps of the measurement process according to one of the embodiments described above.

### BRIEF DESCRIPTION OF THE DRAWINGS

Embodiments of the invention will be described below with reference to the drawings, briefly described below:

FIG. 1 represents the main elements of a non-invasive sensor 1 according to the invention, the non-invasive sensor 1 in this case being positioned in contact with the target stratified environment 2.

FIG. 2 is a simplified representation of the various transmitted and reflected thermal waves generated in a bilayer stratified environment in response to laser irradiation.

FIGS. 3a and 3b show the amplitude of the thermal wave generated in response to irradiation as a function of modulation frequency for various glucose concentrations, for two different target stratified environment.

FIG. 4 shows a typical model of skin-type of a target stratified environment.

FIG. 5 shows the steps of a measurement procedure according to the invention in a particular embodiment, where a patient's interstitial blood sugar is to be measured.

FIG. 6 shows the steps implemented by simulation module 15 to simulate a photoacoustic signal that would be detected by a given photoacoustic cell in response to irradiation of a target stratified environment modeled using the parameters of model configuration CMk according to the irradiation parameters of irradiation case Ij.

FIG. 7 shows how an inverse model works.

FIG. 8a shows the values predicted by the non-invasive sensor as a function of the actual values, in the case where the target stratified environment is a patient's skin, modeled by a bilayer model (stratum corneum, epidermis).

The thickness of the upper layer (modeling the stratum corneum) is equal to 14 mm, and the thickness of the lower layer (modeling the epidermis) is considered infinite. Blood sugar is zero in the upper layer. The upper layer is 20% water and the lower layer is 60% water. Irradiation is performed at the frequencies foptim=1580 Hz, fnod1=950 Hz and fnod2=4750 Hz corresponding to this model configuration.

FIG. 8b shows the values predicted by the non-invasive sensor as a function of the actual values, in the case where the target stratified environment is a patient's skin, modeled by a bilayer model (stratum corneum, epidermis). The thickness of the upper layer (modeling the stratum corneum) is equal to 18 mm, and the thickness of the lower layer (modeling the epidermis) is considered infinite. Blood sugar is zero in the upper layer. The upper layer is 20% water and the lower layer is 60% water. Irradiation is carried out at frequencies foptim=1580 Hz, fnod1=950 Hz and fnod2=4750 Hz, not corresponding to this model configuration (layer thickness equal to 18 mm) but corresponding to the model configuration used for FIG. 8a.

FIG. 8c shows the values predicted by the non-invasive sensor as a function of the actual values, in the case where the target stratified environment is a patient's skin, modeled by a bilayer model (stratum corneum, epidermis). The thickness of the upper layer (modeling the stratum corneum) is equal to 18 mm, and the thickness of the lower layer (modeling the epidermis) is considered infinite. Blood sugar is zero in the upper layer.

The upper layer is 20% water and the lower layer is 60% water. Irradiation is performed at the frequencies foptim=1000 Hz, fnod1=600 Hz and fnod2=1300 Hz corresponding to this model configuration.

### DETAILED DESCRIPTION

The invention relates to a non-invasive sensor 1 for one or more parameters of a target stratified environment 2, the structuring of which may possibly change over time. The target stratified environment 2 can be, by way of example, a tissue of a human or animal organism, such as skin. The parameters to be measured are hereinafter referred to as “parameters of interest”.

In particular, a parameter of interest may be a physiological parameter in the case where the stratified environment is a human or animal tissue.

For example, the physiological parameter to be measured is blood sugar, in particular interstitial blood sugar. It could also be a question of measuring the water content in a particular layer of the skin, or the lactate concentration of a particular layer. These examples are not limitative.

The non-invasive sensor 1 may be portable and may enable continuous monitoring of the parameter(s) of interest.

The non-invasive sensor 1 may be based on photoacoustic detection or photothermal detection. The measurement method is particularly suitable for improving the accuracy of a non-invasive sensor based on indirect photoacoustic detection, in which the non-invasive sensor 1 detects an acoustic wave generated in the fluid surrounding the target stratified environment 2 in response to irradiation, while controlling its energy consumption, or for reducing the sensor's energy consumption while controlling its accuracy. However, it is entirely possible to implement this process on a non-invasive photothermal based sensor. To simplify understanding, the example of indirect photoacoustic detection will be described in more detail hereinafter, but generalization to a photothermal based sensor will be made without difficulty.

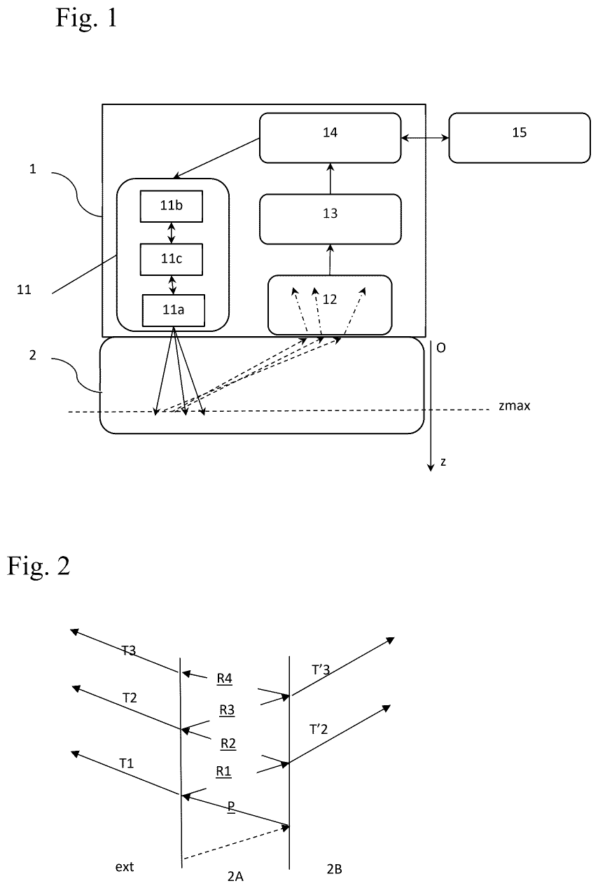

The non-invasive sensor 1 is shown schematically in FIG. 1 and comprises
an irradiation device 11 comprising a light source 11a, an intensity modulation device of this light source 11b, a control device 11c at least one modulation frequency at which the intensity modulation device 11b modulates the intensity of a light emitted by the light source 11a;
at least one photoacoustic detection cell 12 detecting a signal generated in response to irradiation of a target stratified environment 2 by the emitted light, for example directly or indirectly detecting a thermal wave propagating in a target stratified environment 2;
a signal processing module 13 configured to receive and process data from the at least one detection cell 12;
an irradiation parameter and calibration model adaptation module 14;
in some embodiments, a simulation module 15, remote or embedded with the other elements of the non-invasive sensor 1.

In a particular embodiment, the light source 11a emits an intensity-modulated laser beam at at least one particular wavelength towards the target stratified environment 2.

The at least one wavelength can be chosen as a function of one of the parameters of interest. By way of example, the wave number 1034 cm⁻¹, corresponding to a glucose absorption peak, may be relevant if the non-invasive sensor 1 is a blood sugar sensor.

In particular, the light source 11a may be a light-emitting diode (LED) or a laser chip. Alternatively, light source 11a may comprise a quantum cascade laser (QCL) emitting in the mid-infrared region (MIR-QCL), an ICL laser (“Interband Cavity Laser”), an internal or external cavity laser, a GaSb laser. These examples are not limitative. The light source 11a can be chosen according to the target stratified environment 2 and/or the parameters of interest.

The non-invasive sensor 1 may comprise several different and/or identical light sources 11a. The non-invasive sensor 1 also comprises the circuits associated with the light source(s) 11a and at least one control device 11c configured to control the frequency at which at least one intensity modulation device 11b modulates the intensity of at least one light source 11a, so that the intensity of the light emitted by the light source 11a is modulated at an adjustable modulation frequency.

The intensity modulation frequency of a given light source 11a is referred to as fmod in the following.

The light source 11a can be intensity-modulated by any known electrical or mechanical means.

The light irradiating the target stratified environment 2 may be emitted continuously or pulsed by a given light source 11a.

The light incident on the target stratified environment 2, emitted by the intensity-modulated light source(s) 11a, propagates to the target stratified environment 2 and then through the target stratified environment 2 (symbolized by solid arrows in FIG. 1). It is then progressively absorbed by the various constituents of this target stratified environment 2, over a characteristic depth, noted zmax, which depends on the structure of the target stratified environment 2 and its physico-chemical composition.

Absorption of the light energy causes local heating of the target stratified environment 2. As a result, a thermal wave with a frequency equal to the modulation frequency of the light source propagates through the target stratified environment 2 (symbolized by the dotted arrows in FIG. 1), in particular towards the surface of the target stratified environment 2. This thermal wave gives rise in the gaseous environment outside the target to a pressure wave of the same frequency, which propagates in this fluid environment, for example gaseous, surrounding the target stratified environment 2 and in particular in the photoacoustic detection cell 12 (phenomenon symbolized by alternating dotted arrows on FIG. 1). At this stage, the interference phenomenon of interest to us is not described. It will be described later. In the case of photoacoustic detection, the detection cell 12 comprises a chamber filled with a gas (e.g. air) through which the acoustic wave propagates. The detection cell 12 also comprises one or more suitable sensors placed in this chamber, for example facing the target stratified environment 2. For example, one or more electroacoustic sensors configured to convert the pressure of the acoustic wave into an electrical signal, such as a microphone or a piezoelectric transducer.

Each electroacoustic sensor is functionally connected to the signal processing module 13. The signal processing module 13 may comprise an analog-to-digital converter configured to convert the analog electrical signal from the electroacoustic sensor into a digital signal. The signal processing module 13 may comprise a synchronous detection device adapted to demodulate and extract the signal of interest from the detected signal.

The signal processing module 13 optionally comprises an operational amplifier operatively connected to the analog-to-digital converter and configured to amplify the electronic signal derived from the acoustic response of the target stratified environment 2 transmitted by an electroacoustic sensor.

In example embodiments, the analog-to-digital converter is operatively connected to a digital signal processor for digital signal processing.

The non-invasive sensor 1 according to the invention further comprises an adaptation module 14 for the irradiation parameters and possibly the calibration model, as well as a simulation module 15. These two elements will be described later, after having specified the origin of the technical problem to be solved, namely to provide a non-invasive sensor 1 based on photoacoustic or photothermal detection of controlled precision and/or consumption by taking into account interference phenomena.

### THERMAL Wave Interference Phenomenon

Since the target stratified environment 2 comprises at least two overlapping layers with different physico-chemical characteristics, it has at least one interface at which the incident wave can be reflected.

In a simple case, shown in FIG. 2, in which the target stratified environment 2 comprises two layers 2A and 2B, the upper layer 2A is in contact with the external environment at the 2A/ext interface and with the lower layer at the 2A/2B interface.

The upper layer 2A is, for example, the stratum corneum of a patient's skin and the lower layer 2B is, for example, the epidermis of a patient's skin in the case of a non-invasive sensor 1 adapted for blood sugar measurement.

Even in such a simple case as this, the extensive description of the thermal wave generated in the target stratified environment 2 requires the use of complex models, such as the so-called “Hu model”, described in Hu, Hanping et al. “Generalized theory of the photoacoustic effect in a multilayer material.” Journal of Applied Physics 86 (1999): 3953-3958. This model implicitly includes some thermal interference effects, but does not take into account interference due to multiple reflections at interfaces. Other models propose to take these multiple reflections into account, notably the one described by Cao, J. (2000); Interferential formulization and interpretation of the photoacoustic effect in multilayered cells; Journal of Physics D: Applied Physics, 33(3), 200

Whatever the model chosen, the interference phenomenon has a significant impact on the amplitude of the detected signal and consequently on the accuracy of the measurement. This impact depends on the structuring of the target stratified environment 2.

A rough understanding of this impact can be gained from FIG. 2, which is for educational purposes only. In this case, a so-called “primary” thermal wave P is generated by the irradiation (symbolized by the dotted arrow) at the 2A/2B interface. This primary wave P propagates towards the 2A/ext interface, where it is partially reflected, giving rise to a first wave transmitted to the external environment T1 and a reflected wave R1. The reflected wave R1 may itself be reflected at the 2A/2B interface, giving rise to a transmitted wave T′2 and a reflected wave R2. The reflected wave R2 will itself give rise, after propagation towards the 2A/ext interface, to a second wave transmitted into the external environment T2 and a reflected wave R3, and so on.

All the waves transmitted into the external environment T1, T2, . . . , are likely to interfere with each other, so that the amplitude of the acoustic wave formed in the external environment in response to irradiation is affected. In this case, we can show that the amplitude of the thermal wave at the 2A/air interface has the expression:

$$θ = {T}_{2 A / ext} {R}_{2 A / 2 B} \frac{1}{1 - {R}_{2 A / 2 B} {R}_{2 a / ext} exp ( - 2 ( 1 + j ) {l}_{2} \sqrt{\frac{ω}{2 {α}^{2}}} )} \qquad Formule 1$$

$$\text{Where:}$$

$${R}_{2 A / 2 B} = \frac{{ε}_{2 A} - {ε}_{2 B}}{{ε}_{2 A} + {ε}_{2 B}} is the thermal reflection coefficient at the 2 A / 2 B interface$$

$${R}_{2 A / e x t} = \frac{{ε}_{2 A} - {ε}_{e x t}}{{ε}_{2 A} + {ε}_{e x t}} is the thermal reflection coefficient at the 2 A / ext interface$$

$${T}_{2 A / ext} = \frac{2 {ε}_{ext}}{{ε}_{2 A} + {ε}_{ext}} = is the thermal transmittance at the 2 A / ext interface$$

$${α}_{2} is the thermal diffusivity of environment 2 A$$

$${l}_{2} is the thickness of layer 2 A$$

$${ε}_{2 A} , {ε}_{2 B} , {ε}_{ext} are the respective thermal effusivities of layer 2 A , layer 2 B and the external environment$$

The thermal effusivity and thickness of layer 2A can, in the case of our target stratified environment 2, vary over time. In particular, in the case where the target stratified environment 2 is the skin and the non-invasive sensor 1 is a blood sugar sensor, the thickness of the skin layers, their water concentrations and their glucose concentrations, which are likely to affect the different thermal effusivities, thus change over time, so that at the same detection site, the photoacoustic or photothermal signal is affected.

It is therefore understood that the accuracy and sensitivity of the measurement of the parameter of interest, for example blood sugar, on the basis of the photoacoustic and photothermal signal depends on the information available concerning these interference phenomena.

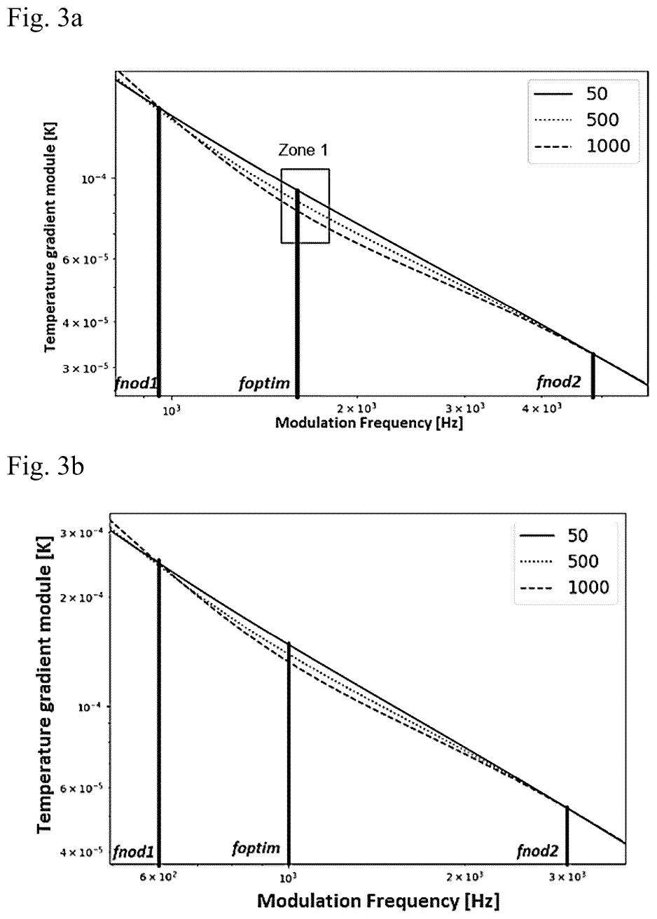

We emphasize here the difficulty of accessing this information in the case of an evolving target stratified environment 2, where the characteristics of the different strata change over time. To solve this problem, the inventors have developed a simulation module 15 which will be described in detail below and which, among other things, simulates the photothermal signal associated with an irradiation whose characteristics (power, wavelength(s), modulation frequency(ies), etc.) are known, the target stratified environment 2 also being modeled by a multilayer environment whose characteristics (thickness, composition, etc.) are known, on the basis of a multiphysics analytical model that at least implicitly takes thermal interference phenomena into account. This simulation module 15 has enabled extensive in silico experiments to be carried out, some of the results of which are shown in FIGS. 3a and 3b. In FIG. 3a, the target stratified environment 2 is the skin, modeled by a bilayer model (stratum corneum, epidermis). The thickness of the upper layer (modeling the stratum corneum) is equal to 14 mm, that of the lower layer (modeling the epidermis) is equal to 10 mm.

layer (modeling the epidermis) is considered infinite. Blood sugar is zero in the upper layer. The upper layer is 20% water and the lower layer is 60% water.

The skin model is irradiated (simulated) with an intensity-modulated 1034 cm⁻¹ laser at an intensity modulation frequency plotted on the x-axis. The amplitude of the simulated thermal response wave generated in response to irradiation is plotted on the ordinate as a function of the modulation frequency for different blood sugar values of lower-layer (50 mg/L; 500 mg/L and 1000 mg/dL).

Unexpectedly, the inventors have observed the existence of so-called “nodal” modulation frequencies in the vicinity of which the amplitude of the thermal wave is independent of the parameter of interest, to be precise the blood sugar.

In the case of FIG. 3a, two nodal modulation frequencies can be observed, namely fnod1=950 Hz and fnod2=4,750 Hz, but there are others outside the modulation frequency range shown in FIG. 3a. These nodal modulation frequencies can be explained by the phenomenon of thermal wave interference within the target stratified environment 2.

Nodal modulation frequencies correspond to a response wave generated in the environment whose amplitude is weakly correlated to the parameter of interest, or even independent of the parameter of interest (in this case: interstitial blood sugar), so the signal detected at such a modulation frequency cannot be used to determine this parameter of interest. In the case of FIG. 3b, the target stratified environment 2 is the skin, modeled by a bi-layer model (stratum corneum, epidermis). The thickness of the upper layer (modeling the stratum corneum) is this time equal to 18 mm, while that of the lower layer (modeling the epidermis) is considered infinite. Blood sugar is zero in the upper layer. The upper layer is 20% water and the lower layer is 60% water.

The skin model is irradiated (simulated) with an intensity-modulated 1034 cm⁻¹ laser at a modulation frequency plotted on the x-axis. The amplitude of the simulated thermal response wave generated in response to irradiation is plotted on the ordinate as a function of the modulation frequency for different blood sugar values of lower-layer (50 mg/L; 500 mg/L and 1000 mg/L).

Nodal modulation frequencies are again observed, in this case fnod1=600 Hz and fnod2=3000 Hz, but these nodal modulation frequencies are different from those obtained in the situation of FIG. 3a, as expected due to the change in thickness of layer 2A from 14 mm to 18 mm.

Generally speaking, the extensive in silico experiments carried out by the inventors have therefore enabled them to observe the following facts:
there are nodal modulation frequencies, in the vicinity of which the amplitude of the thermal wave generated in response to irradiation is weakly correlated to the parameter of interest, or even independent of the parameter of interest. Note that if the scale of FIGS. 3a and 3b is enlarged, it is not really a question of a single nodal modulation frequency, but of a restricted range of frequencies in which the thermal amplitude varies very little when the parameter of interest varies;
the values of the nodal modulation frequencies depend on the characteristics of the target stratified environment 2, namely the thicknesses and compositions of the various layers that make it up.

It is very difficult to carry out sufficient in vivo observations to obtain such curves. These observations have therefore only been made possible by the simulator developed by the inventors. These observations are therefore new and make it possible to envisage a non-invasive sensor 1 that takes account of interference phenomena.

On the basis of these observations, the inventors have designed a non-invasive sensor 1 and a method for detecting a parameter of interest that is improved on the prior art, which will now be described in greater detail.

In the non-invasive sensor 1, nodal modulation frequencies can be taken advantage of in the context of the non-invasive sensor 1 thanks to the adaptation module 14 that characterizes it and, in a particular embodiment to the simulation module 15.

### Adaptation Module 14

Adaptation module 14 is a computerized device comprising at least one processor:
which can exchange information where appropriate with the simulation module 15 described below;
which can receive information from the detection cell 12 and/or the signal processing module 13 if applicable;
and which can transmit information to the control device(s) 11c of at least one irradiation parameter of the light source 11a (e.g. a modulation frequency at which the intensity modulation device 11b modulates the intensity of a light emitted by the light source 11a, the wave number of a light emitted by the light source 11a, the light power of the light source 11a, . . . ).

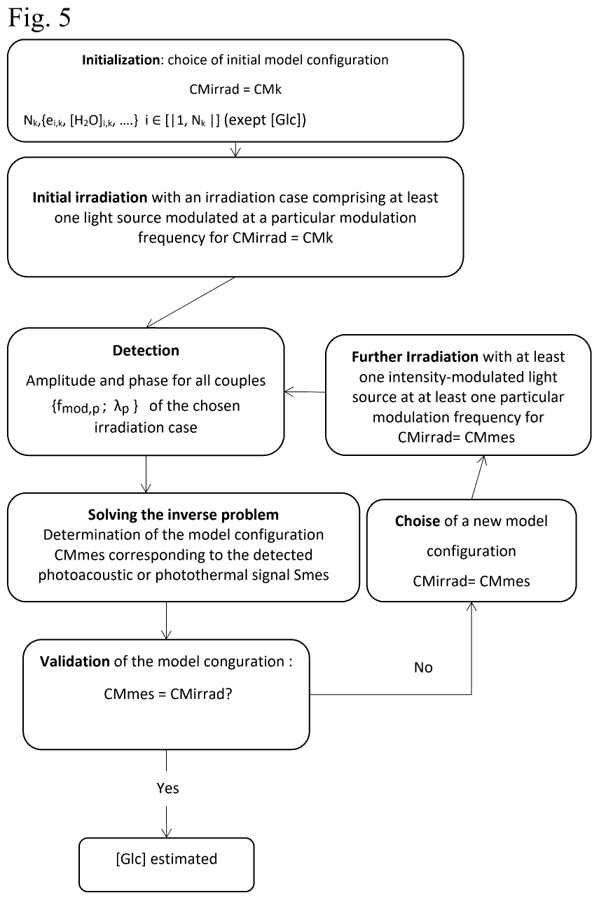

The steps implemented by a non-invasive sensor 1 comprising an adaptation module 14 for a measurement of the parameter of interest are schematically shown in FIG. 5 in a particular embodiment and in the case where the target stratified environment 2 is the skin and the parameter of interest is interstitial blood sugar.

The non-invasive sensor 1 further comprises a memory for storing a database of model configurations, a nodal frequency chart and one or more inverse models which are described below.

This storage memory can be distributed and/or shared in/with the adaptation module (14) and/or the simulation module (15).

The adaptation module (14) carries out the process steps for selecting the irradiation parameters for the measurement on the basis of the nodal frequency chart, and for determining the most suitable, i.e. the most accurate, inverse model for calculating the measured parameter from the signal detected for that particular measurement. We will describe these steps after describing the steps for generating the database of model configurations and the nodal frequency chart.

The model configuration database and the nodal frequency chart can be generated from a simulation module 15, embedded in sensor 1 or remote. If the simulation module is remote, the non-invasive sensor 1 includes communication means for the simulation module 15 and the adaptation module 14 to exchange data.

### Simulation Module 15

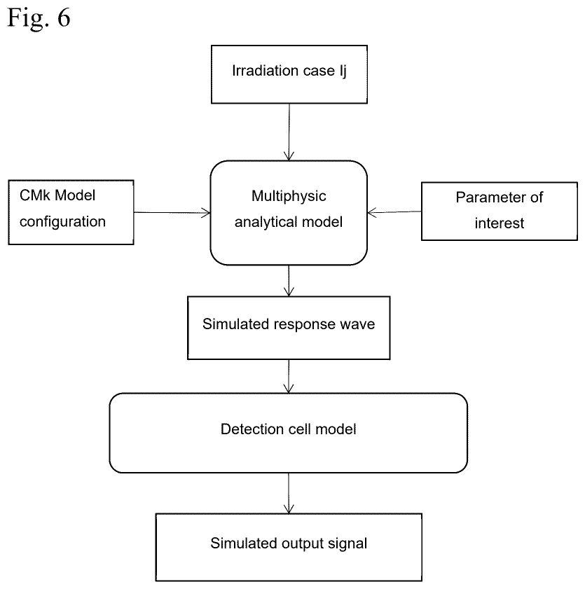

The simulation module 15 is a computerized device configured to generate a set of model configurations each corresponding to (or alternatively modeling or describing) a particular state of the target stratified environment 2, a set of irradiation cases (each comprising at least one irradiation parameter such as light power, wavelength and modulation frequency) of the target stratified environment 2 and the photoacoustic signals (or where appropriate photothermal) theoretically detected in response to each irradiation case for each model configuration CMk of the target stratified environment 2 from analytical models of the target stratified environment 2 and the photoacoustic (or photothermal, as the case may be) detection cell 12, as shown in FIG. 6.

This simulation module 15 is particularly relevant in our case, where the target stratified environment 2 is evolving over time and/or stratified. In this case, the target stratified environment 2 adopts different actual configurations over time (one or more concentrations vary within one or more layers of the target stratified environment 2, one or more dimensions such as, for example, the thickness of one of the layers of the target stratified environment 2 vary), each of which can be modeled by a particular model configuration.

### a) Multiphysics Analytical Model of Target Stratified Environment 2

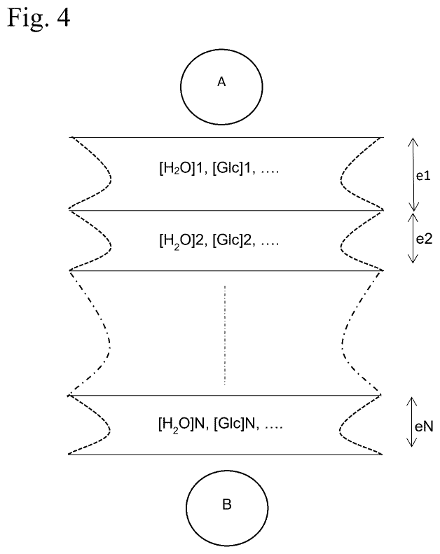

The stratified target medium 2 to be analyzed is modeled as shown in FIG. 4. The target medium 2 separates an outer medium A from an inner medium B and is composed of a succession of N layers whose interfaces are, for example, assumed to be locally planar. Each layer i (i∈ [symbol] 1, N [symbol] ) is described by the parameter(s) of interest and a number of explicit parameters (referred to as level 1 parameters as their values will be provided as input to the simulation module 15 for each simulation) appropriate for the target stratified environment 2 under consideration. For example, if the target stratified environment 2 is the skin, and the aim is to measure a glucose concentration in a layer j of this target environment 2, the target environment 2 will be described by its number of layers N, the concentration [Glc]j of interest, and each layer i can be described for the modeling by its thickness ei, its water concentration [H2O]i. Glucose concentrations [Glc]i for if j may also be taken into account. In this case, the values of ei (i∈ [symbol] 1, N [symbol] ), the [H2O]i concentrations (i∈ [symbol] 1, N [symbol] ) and [Glc]i concentrations for (i∈ [symbol] 1, N [symbol] i≠j) are level 1 parameters.

The list of level 1 parameters can be extended if more precise modeling is desired. In particular, the concentrations of other skin components such as fats, lactate, oxygen, etc., can be included in the list of level 1 parameters describing a skin layer.

Again for the skin example, it may be possible to take account of skin color, patient age, or any other anthropometric parameter, so as to expand or restrict the space of possible models. Non-explicit parameters of the target stratified environment model 2 (called level 2 parameters because they are not provided as input to the simulation module 15) can be calculated by means of analytical models. For example, the thermal conductivity, thermal capacity, density or absorption coefficient at each wavelength of each layer of the target stratified environment 2 can be deduced from level 1 parameters and known equations. As the parameter of interest plays a special role, it is not included in the list of level 1 parameters. Depending on the process step, this parameter of interest may or may not be known: its value is known in order to carry out simulations using the simulation module 15, but it is of course unknown in the case of an actual measurement with the non-invasive sensor 1.

The number of layers N of the target stratified environment 2 can also be a model variable. Again in the skin example, depending on the physiological situation, N may be greater than or equal to two. Thus, for certain physiological situations, the skin may be correctly described by two layers, the first corresponding to the stratum corneum, whose glucose concentration may, for example, be low, and the second to the rest of the skin, the glucose concentration of the second layer being assimilated to the interstitial glucose concentration to be measured.

For other situations, a model with three or more layers may be more appropriate. In the latter case, the water concentration of a layer may, for example, increase with the depth of the layer. N may therefore not be a constant.

In the case of the skin, the external medium A is generally the atmosphere surrounding the patient, which also fills the photoacoustic detection cell.

Simulation module 15 implements a multiphysics analytical stratified environment 2 model based, for example, on physical and/or chemical equations such as, by way of non-limiting example, the Beer-Lambert equations for optical absorption and the thermodynamic equations of heat (Fourier law and conservation laws).

A model configuration CMk (k positive integer) of target stratified environment 2 corresponds to (or models) a particular state of the given target stratified environment 2. This particular state is assumed to be correctly represented by giving the number of layers N and the values of the level 1 parameters for each layer.

For each CMk model configuration, the multiphysics analytical model makes it possible, if the parameter of interest is also known, to simulate the thermal wave generated at the layer 1/outer environment A interface (interface 1/A) in response to irradiation by a light source 11a whose irradiation parameters are known, namely for example the modulation frequency fmod, the wavelength 1 and the power flux density.

Alternatively, the multiphysics analytical model can be used to simulate the pressure wave generated in the external environment A.

In both cases, the signal obtained at the output of a processor implementing the multiphysics analytical model is called the “simulated response wave”.

The simulated response wave can be provided as input to a processor implementing the detection cell model.

### b) Detection Cell Model

The non-invasive sensor 1 based on photoacoustic or photothermal detection comprises a photoacoustic detection cell 12 (respectively photothermal) configured to detect and analyze the pressure wave (respectively thermal wave) generated in the external environment A when the thermal wave generated in the target stratified environment 2 in response to irradiation reaches the interface 1/A.

The entire detection cell 12 can be modeled analytically. From a response wave simulated by the multiphysics analytical model, which would theoretically be received at the input of the detection cell 12, the detection cell 12 model predicts the output signal of the detection cell 12.

Various models can be envisaged.

For example, in the case of indirect photoacoustic detection, the parameters of the model of the detection cell 12, hereinafter referred to as cell parameters, may include its dimensions (i.e. vent size, cell height, etc.), thermodynamic state parameters (temperature, atmospheric pressure, relative or absolute humidity, etc.). In particular, the photoacoustic detection cell can be modeled by means of an equivalent RLC circuit. By way of example, a model derived from the model described in Dehe, Alfons et al. “The Infineon Silicon MEMS Microphone.” (2013) may be suitable.

It is possible to include in this photoacoustic detection cell model a model of the signal processing step carried out by the signal processing module 13 if applicable, so as to generate, from each simulated response wave generated by the processor implementing the analytical multiphysics model, the signal theoretically obtained at the photoacoustic detection cell output (and if applicable after signal processing by the signal processing module 13) which corresponds to it.

The processor of simulation module 15 can be configured to implement the photoacoustic detection cell model.

Thanks to the multiphysics analytical model of the stratified target environment 2 and the model of the detection cell 12, a global analytical model is thus available which allows, given the model configuration CMk of the stratified target medium 2 and the irradiation case Ij, and provided that the parameter of interest is also supplied (which can indeed be chosen, since this is a simulation), makes it possible to predict the signal expected at the output of the detection cell 12 or, where applicable, the signal processing module 13. This is shown in FIG. 6.

The multiphysics analytical model is chosen to take account, at least implicitly, of thermal wave interferences. In a particular embodiment, it can take these interferences into account explicitly.

Alternatively, it can take into account all or part of the multiple reflections at the interfaces of the target stratified environment 2.

In summary, the simulation module 15 therefore receives as input the parameters of the model configuration CMk of target stratified environment 2, i.e. the number of layers N of target environment 2 and the level 1 parameters for each layer, as well as the parameter of interest and the irradiation parameters of irradiation case Ij. As an output, the simulation module 15 provides the theoretically expected signal at the output of the detection cell 12 or, where applicable, the theoretically expected processed simulated signal at the output of the signal processing module 13 for the model configuration CMk of target environment 2, referred to as the simulated output signal.

The simulated output signal can be stored in memory in the form of a Fourier spectrum.

The multiplets {target environment CMk model configuration 2, irradiation case Ij, parameter of interest, amplitudes and phases of the simulated output signal components}can be stored in a model configuration database.

### Database of Model Configurations

A large number of model configurations CMk can therefore be generated, possibly automatically and/or randomly, each model configuration CMk corresponding to a number of layers N and a set of level 1 parameters, and optionally to a value or range of values of the parameter of interest, describing a particular situation of the target stratified environment 2 of interest.

For each model configuration CMk, a large number of irradiation cases Ij can be generated, possibly automatically and/or randomly, each irradiation case corresponding to a set of irradiation parameters describing the parameters of the light source(s) 11a used for irradiation. An irradiation case Ij may therefore comprise one or more frequencies for modulating the intensity of one or more lasers, the wavelength of each of these lasers and optionally the power irradiated by each laser.

By means of simulation module 15, the amplitude and phase of each component of the simulated output signal obtained at the output of the processor of simulation module 15 implementing the global analytical model comprising in cascade the multiphysics analytical model and the detection cell model are calculated for each model configuration CMk for each irradiation case Ij, a value of the parameter of interest also being provided.

The irradiation cases Ij may be the same for several different model configurations CMk, and possibly several values of the parameter of interest, or different from one model configuration CMk to another and/or from one value of the parameter of interest to another. Once the simulations have been carried out, all these CMk model configurations, irradiation cases, parameters of interest and associated simulated photoacoustic (or photothermal) signals can be stored in a model configuration database in the form of multiplets {model configuration CMk, irradiation case Ij, parameter of interest, amplitude and phase of simulated output signal components}.

The generation of CMk model configurations and/or Ij irradiation cases may not be completely random.

The generation of CMk model configurations may, among other things, be based on physiological considerations to restrict the space of possibilities to physiologically realistic model configurations. For example, the possible thicknesses of the first skin layer can be limited to the range [8 mm, 40 mm] that is actually observed experimentally, and the water concentrations of this layer can be limited to a restricted range for each thickness, the water concentration of the stratum corneum being correlated with its thickness.

In particular, the generation of irradiation cases can take into account the limitations of the light sources 11a available for a given non-invasive sensor 1 in wavelength and/or power, or the modulation frequency ranges relevant to the type of target stratified environment 2 to be analyzed, or the wavelengths relevant to the parameter(s) of interest.

Alternatively, the database of model configurations may comprise only multiplets {model configuration CMk, irradiation case Ij, parameter of interest, amplitudes and phases of the signal components actually measured}obtained by experiments in real life situations, or comprise both such multiplets obtained in real life situations and multiplets obtained by simulation.

### Inverse Model

One or more artificial intelligence models can be trained in the simulation module 15 from the model configuration database.

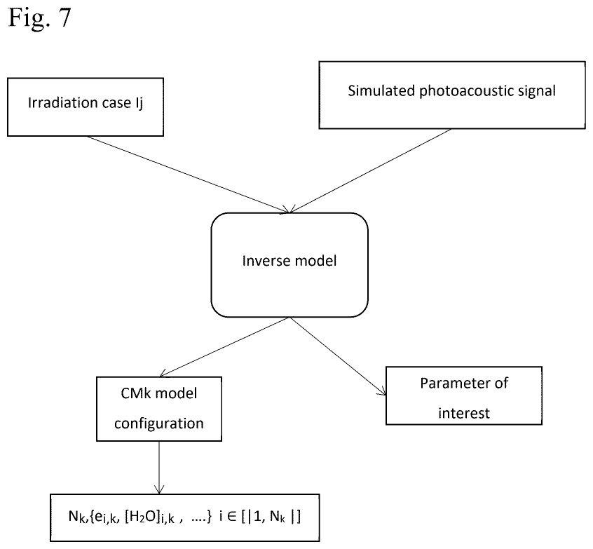

After learning, the artificial intelligence model is able to solve the inverse problem, i.e. to find the parameter(s) of interest and the model configuration CMk of target environment 2, i.e. the number of layers N and the level 1 parameters, knowing the simulated photoacoustic or photothermal signal and the irradiation parameters of the irradiation case, as shown in FIG. 7.

The learned model, hereinafter referred to as the inverse model, can be transmitted to the adaptation module 14 and stored in memory of this model.

Several different inverse models can be learned, with different training sets and/or training rules.

### Nodal Frequency Chart

We have already seen that a large number of model configurations CMk can be generated, possibly automatically and/or randomly, each model configuration CMk corresponding to a number of layers N and a set of level 1 parameters, and optionally to a value or range of values of the parameter of interest, describing a particular situation of the target stratified environment 2 of interest.

Groups GMp of model configurations can therefore be generated, with two model configurations in the same group differing only in the value of the parameter of interest.

For each model configuration CMk, we have also seen that a large number of irradiation cases Ij can be generated, possibly automatically and/or randomly, each irradiation case corresponding to a set of irradiation parameters describing the parameters of the light source(s) 11a used for irradiation.

It is therefore also possible to generate groups GIq of irradiation cases, with two irradiation cases in each group GIq of irradiation cases differing only in the modulation frequency of a light source 11a.

For a group GMp of model configurations CMk which differ from one another only in the value of the parameter of interest, the amplitude curves of the detected signal as a function of the modulation frequency of the light source can be generated by means of simulated irradiations with a given group of irradiation cases GIq, with only the modulation frequency varying from one irradiation case to another. This is how FIGS. 3a and 3b were obtained. From these curves, possibly by means of the processor of the simulation module 15 or the processor of the adaptation module 14, at least one nodal modulation frequency can be determined for this set of model configurations GMp.

It is therefore possible to determine a plurality of multiplets, hereinafter referred to as nodal multiplets. Each nodal multiplet comprises:
characteristics common to all the elements of a given GMp group of model configurations of the target stratified environment (number of layers, layer thicknesses, concentrations . . . excluding the parameter of interest which varies from one element of the GMp group to another;
and a plurality of nodal modulation frequencies associated with this GMp group, for which the acoustic or thermal signal detected by the detection cell in response to irradiation by the light source exhibits a correlation below a predetermined threshold with the parameter of interest.

A nodal frequency chart can therefore be generated from the database of model configurations, for example using simulation module 15.

Once the simulations have been carried out, this nodal frequency chart can be stored in a memory of the simulation module, the chart comprising at least one plurality of nodal multiplets each comprising at least the characteristics common (or the set of irradiation parameters common) to a group of GMp model configurations and a plurality of associated nodal modulation frequencies.

The nodal multiplets may optionally comprise one or more so called particular frequencies, noted foptim, for which the sensor sensitivity is maximum in the modulation frequency range delimited by two (possibly successive) nodal modulation frequencies for the concerned parameter of interest.

Such a particular modulation frequency foptim can be observed in zone 1 shown in FIG. 3a. In one embodiment, a particular modulation frequency is determined by means of the processor of simulation module 15 or adaptation module 14 on the basis of a weighted average of at least two successive nodal modulation frequencies.

At this stage, the elements required to implement the measurement process according to the invention are ready.

It is already understood that, thanks to the nodal frequency chart, it will be possible to choose the irradiation parameters to take account of interference phenomena and thus reduce the number of irradiation modulation frequencies required to satisfy one or more optimization criteria, in particular among an energy consumption criterion, a sensitivity criterion and an accuracy criterion.

The nodal frequency chart can be stored in a memory of adaptation module 14 or simulation module 15 or another memory of non-invasive sensor 1.

Alternatively, the nodal frequency chart is generated from data obtained via in vivo experiments. In this embodiment, the simulation module 15 is not required.

In another embodiment, the nodal frequency chart is generated both from data obtained in silico using the simulation module and from data obtained in vivo.

### Process for Measuring the Parameter of Interest

A particular embodiment of the method for measuring the parameter of interest by means of the non-invasive sensor 1 based on indirect photoacoustic or photothermal detection is shown in FIG. 5. It comprises the following steps:

### a) Initialization:

Adaptation module 14 selects an initial model configuration of target stratified environment 2 for irradiation on the basis of predetermined criteria. For example, in FIG. 5, the initial CMirrad model configuration is the CMk model configuration.

Various options are available for this initialization.

In particular, in the case of interstitial blood sugar measurement from the model configuration database and/or from a database of experimental measurements, an average set of level 1 and blood sugar parameters can be determined for a patient population or for a given patient. This average parameter set corresponds to an initial model configuration CMk of the target stratified environment 2, which has the highest probability of being the most suitable for the forthcoming measurement in the absence of any other information, and in particular in the absence of a measurement history.

### b) Adaptation of Irradiation Parameters

1—From the nodal frequency chart, the processor of adaptation module 14 determines a plurality of nodal modulation frequencies fnod associated with the group GMp of model configurations of the target stratified environment to which the initial model configuration CMirrad belongs;
2—Then it determines at least one particular modulation frequency foptim on the basis of this plurality of nodal modulation frequencies. The at least one particular modulation frequency foptim can be a modulation frequency for which the sensor sensitivity is maximum in the modulation frequency range delimited by two (possibly successive) nodal modulation frequencies for the parameter of interest concerned. In one embodiment, the at least one particular modulation frequency foptim is determined by means of the processor of simulation module 15 or adaptation module 14 on the basis of a weighted average of at least two successive nodal modulation frequencies.
3—And finally, it determines on the basis of these nodal modulation frequencies a particular irradiation case for the model configuration of the target stratified environment 2 CMirrad, which is an irradiation case comprising at least the particular irradiation frequency foptim for the initial model configuration CMirrad. In particular, the particular irradiation case can be denoted Iopt.k since it is optimized with respect to a criterion for taking interference phenomena into account.

### c) First Irradiation:

The target environment 2 is irradiated for the first time, at least one of the light source(s) 11a being configured according to the set of irradiation parameters corresponding to the particular irradiation case.

### d) PA Detection:

The actual photoacoustic signal generated in response to irradiation is detected by mean of the photoacoustic detection cell 12.

If necessary, the signal processing module 13 receives and processes this actual photoacoustic signal and, after processing, transmits it to the adaptation module 14.

### e) Determination of the Parameter of Interest

The processor of adaptation module 14 determines the parameter of interest, in this example blood sugar, on the basis of the detected photoacoustic or photothermal signal.

Several embodiments of step e) are possible.

In particular, the processor of adaptation module 14 can implement a single inverse model. Alternatively, the processor of adaptor module 14 can use an operating law to deduce a blood sugar value from the detected signal.

In the most sophisticated embodiment shown in FIG. 5, step e) can be broken down as follows:

### e1) Solving the Inverse Problem

The processor of adaptation module 14 implementing the learned inverse model(s) (and in particular at least that corresponding to model configuration CMk of target stratified environment 2 used for irradiation) receives as input the actual photoacoustic or photothermal signal and the particular irradiation case selected for irradiation, noted for example Iopt.k, and determines the current target stratified environment 2 model configuration, noted CMmes, as well as the parameter of interest (as shown in FIG. 7).

### e2) Validation of the Model Configuration

The adaptation module 14 compares the current stratified target environment 2 model configuration CMmes with the model configuration CMirrad used for irradiation (model configuration CMk from the initialization step for the first measurement, model configuration CM<possibly different for a subsequent measurement).

Case 1) If the measured stratified target environment 2 model configuration CMmes is identical to the model configuration CMirrad that was used for irradiation, the plurality of nodal modulation frequencies used to determine the particular modulation frequency foptim and consequently the chosen irradiation parameters, i.e. the particular irradiation case, were adapted to the physiological situation being measured. In other words, the particular irradiation case selected was optimized to take account of interference phenomena for the current physiological situation (which, it should be remembered, is not known a priori and evolves over time), as was the inverse model used to determine the parameter of interest. Consequently, the parameter of interest determined in step e1) by adaptation module 14 is the result of the measurement.

Case 2) If the measured target stratified environment model configuration 2 CMmes is different from the model configuration CMirrad, the adaptation module processor then searches the nodal frequency chart for a new plurality of nodal modulation frequencies, this time corresponding to the group of model configurations GMp′ to which the new target stratified environment model configuration CMmes belongs, and determines, on the basis of this plurality of nodal modulation frequencies, a particular new irradiation case which comprises at least one modulation frequency foptim determined on the basis of the plurality of nodal modulation frequencies for the target stratified environment model configuration 2 CMmes. The particular new irradiation case is, for example, denoted Iopt. <, as it is optimized to take account of interference phenomena for the physiological situation. The adaptation module processor 14 transmits the corresponding parameters to the irradiation device 11. The target stratified environment 2 is then irradiated again (subsequent irradiation) according to the irradiation parameters of irradiation case Iopt. <. Then steps d) for detecting and, if necessary, processing the photoacoustic or photothermal signal and e1) for solving the inverse problem are reiterated.

The process may also include a step e2) for validating the model configuration.

Alternatively, the process may also comprise a reiteration of step e2) for validating the model configuration.

In any case, in step e), the model configuration of the target stratified environment 2 being CMmes may be different from the model configuration CMirrad. In fact, CMirrad has been chosen
either according to the initialization criteria (in which case CMirrad is the “average” model configuration, particularly in the absence of measurement history on the patient. This is the case for the first irradiation);
or on the basis of the previous measurement (CMirrad is then the most suitable model configuration for the patient, knowing the measurement result following the previous irradiation).

CMirrad is therefore validated using additional information acquired from the current irradiation, namely the signal detected by photoacoustic detection cell 12. If, by chance, CMmes=CMirrad, no further irradiation is required and the value of the parameter of interest is indeed the most accurate that could be obtained, but the model configuration validation step has provided additional information, namely confirmation that the measurement accuracy is indeed maximum for this case.

In the case where CMmes is different from CMirrad, adaptation of the irradiation parameters and of the model configuration makes it possible to increase the accuracy of the measurement at the cost of at least one additional irradiation, but with energy consumption still under control, and thanks to the second validation step, to confirm that the accuracy of the measurement is indeed maximum. If the validation step is allowed to be repeated (as shown in FIG. 5), the process output acquires the additional information that the measurement accuracy is indeed maximum.

In general, in this case, the measurement result of the parameter of interest is obtained after the first irradiation or after two irradiations. However, to ensure that the process converges and/or to limit energy consumption, the number of steps required to validate the model configuration can be limited.

In all cases, therefore, it can be seen that selecting an irradiation case on the basis of the nodal frequency chart makes it possible to limit the number of modulation frequencies and wavelengths used for irradiation, retaining only those values that provide non-redundant information on the current physiological target environment 2 model configuration and are sufficient to obtain the desired measurement accuracy and/or optimal for limiting sensor power consumption to a predetermined value.

In particular, the particular modulation frequency or frequencies of the sources are selected to take account of interference phenomena: a particular modulation frequency foptim is a modulation frequency for which the measurement sensitivity is greatest between two nodal modulation frequencies. Choosing an irradiation case with at least one particular modulation frequency increases the sensor's sensitivity without increasing its power consumption.

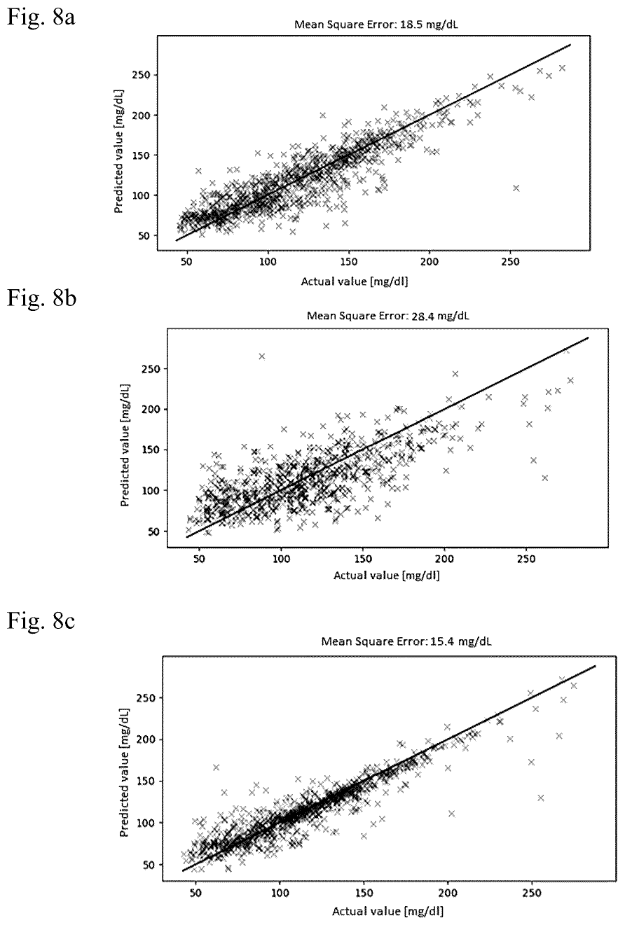

This can be seen from FIGS. 8a, 8b and 8c: to obtain these three figures, three successive situations were simulated. For all three figures, the target stratified environment 2 is a patient's skin, modeled by a bilayer model (stratum corneum, epidermis). The thickness of the top layer (modeling the stratum corneum) is equal to 14 mm for FIG. 8a, and it is assumed that an event varies this thickness so that it evolves and becomes equal to 18 mm when the measurements for FIG. 8b and FIG. 8c are taken. The thickness of the lower layer (modeling the epidermis) is considered infinite. Blood sugar is zero in the upper layer. The upper layer is 20% water and the lower layer is 60% water.

The nodal frequency chart is used by the processor of adaptation module 14 to determine a plurality of nodal frequencies for the initial situation (stratum corneum thickness equal to 14 mm) and to determine the irradiation case to be used for this situation on the basis of these nodal frequencies. In this case, to obtain the measurement points shown in FIG. 8a, the skin model is irradiated (in a simulated way) with a laser of wave number 1034 cm-1 modulated in intensity at the three modulation frequencies foptim=1580 Hz, fnod1=950 Hz and fnod2=4750 Hz determined from FIG. 3a.

Similarly, the nodal frequency chart enables the processor of the adaptation module 14 to determine a plurality of nodal modulation frequencies for the final situation (stratum corneum thickness equal to 18 mm) and to determine the irradiation case to be used for this situation on the basis of these nodal frequencies. In this case, to obtain the measurement points shown in FIG. 8c, the skin model is irradiated (in a simulated way) with a laser of wave number 1034 cm-1 modulated in intensity at the three modulation frequencies foptim=1000 Hz, fnod1=600 Hz and fnod2=3000 Hz determined from FIG. 3b.

In contrast, to obtain the measurement points shown in FIG. 8b, the skin model is irradiated with a laser of wave number 1034 cm-1 intensity modulated at the three modulation frequencies specifically adapted to the skin model shown in FIG. 8a, namely foptim=1580 Hz, fnod1=950 Hz and fnod2=4750 Hz.

We can see that in the case where the irradiation case is selected on the basis of nodal modulation frequencies adapted to the current physiological situation, i.e. for FIGS. 8a and 8c, the mean square deviation (RMSE) between the predicted and actual values is acceptable (18.5 mg/dL and 15.4 mg/dL respectively). On the other hand, if nodal modulation frequencies are inappropriate (as in FIG. 8b), the mean square deviation (RMSE) between predicted and actual values deteriorates sharply, reaching 28.4 mg/dL in this case.

It is therefore clear that, while the choice of a limited number of modulation frequencies on the basis of the nodal modulation frequencies makes it possible to control the power consumption of the non-invasive sensor, the adaptation of the nodal modulation frequencies (on the basis of which the particular modulation frequencies are determined) to the current physiological situation also makes it possible to control the accuracy of the non-invasive sensor 1, for example in such a way that the mean square deviation or any other quantity making it possible to quantify the accuracy is below a predetermined threshold.

The detection method can include the selection for each irradiation case of a single particular modulation frequency foptim determined on the basis of a plurality of nodal frequencies, for example by means of an arithmetic or weighted average. In particular, in the case of a weighted average, greater weight can be assigned to the lowest nodal modulation frequency. This arrangement takes into account the fact that the amplitude of the photothermal signal decreases with the modulation frequency of the incident light wave intensity.

By way of example, in the particular case of a photothermal measurement in the thermal piston regime described in Kottmann, J. et al. (2012); Glucose sensing in human epidermis using mid-infrared photoacoustic detection; Biomedical optics express, 3(4), 667-680, the amplitude of the photothermal signal is proportional to the inverse of the modulation frequency of the incident light wave. Consequently, particularly in this case, a particular modulation frequency foptim can be the barycenter of two nodal modulation frequencies, each assigned a weight equal to its inverse. If we denote f1 and f2 as two successive nodal modulation frequencies, the particular modulation frequency can therefore be calculated as follows:

$$f optim = \frac{\frac{1}{f 1} f 1 + \frac{1}{f 2} f 2}{\frac{1}{f 1} + \frac{1}{f 2}} = 2 \frac{f 1 f 2}{f 1 + f 2} \qquad Formula 2$$

In another embodiment, particularly suited to the case of a closed photoacoustic detection cell, to take account of the frequency response of the detection cell, a particular modulation frequency foptim can be the barycenter of two successive nodal modulation frequencies each assigned a weight equal to its inverse raised to the power 3/2.

Further weights can be chosen, for example to take account of the frequency responses of one or more other elements in the measurement chain.

Alternatively, for each irradiation, the irradiation case selected in the process can include, in addition to a particular foptim modulation frequency, at least one nodal modulation frequency. Indeed, while the detected signal corresponding to the nodal modulation frequency does not carry information concerning the parameter of interest, it does carry information concerning the structuring of the stratified environment 2, independently of this parameter of interest. Selecting both a particular modulation frequency foptim and a nodal modulation frequency fnod can therefore lead to the inverse problem being solved with greater efficiency and accuracy than any other selection of two modulation frequencies.

In particular, as has been done for the examples in FIGS. 8a, 8b and 8c, it is possible to select one or both nodal modulation frequencies closest to the optimum frequency foptim determined.

It is also possible to select two or more different particular modulation frequencies.

For example, if the main optimization criterion is energy consumption, i.e. if the non-invasive sensor is powered by a battery that reaches a predetermined lower charge threshold, it is possible to select only one modulation frequency for irradiation, namely a particular foptim modulation frequency.

If the energy consumption criterion is less important and irradiation at two different modulation frequencies is permitted, a particular modulation frequency foptim and the lowest of the nodal modulation frequencies used to determine foptim. Indeed, it is at this lowest nodal modulation frequency that the signal amplitude will be the highest and therefore the accuracy the best.

If the aim is to optimize sensor sensitivity or accuracy with even lower energy consumption, a specific modulation frequency foptim and two nodal modulation frequencies can be used for irradiation. modulation frequencies to determine foptim, and optionally also a second, third, etc., special modulation frequency.

Finally, the invention relates to a computer program comprising instructions which lead the non-invasive sensor 1 according to one of the preceding embodiments to execute the steps of the process according to any of the embodiments described above.

### LIST OF REFERENCE SIGNS

1: non-invasive sensor based on indirect photoacoustic detection
11: irradiation device
11a: light source
11b: light source intensity modulation device 11a
11c: device for controlling the intensity modulation frequency fmod of light source 11a
12: detection cell
13: signal processing module
14: adaptation module
15: simulation module
2A: first layer of target stratified environment
2B: second layer of target stratified environment

1. A method of measuring a parameter of interest in a stratified target environment by means of a non invasive sensor based on photoacoustic detection or photothermal detection, wherein the method comprises:
a) a sensor is provided comprising
a light source,
a device for controlling a plurality of irradiation parameters of the light source, the plurality of irradiation parameters comprising at least one frequency for modulating the intensity of the light source,
a detection cell configured to detect an acoustic or thermal signal,
a memory in which is stored a nodal frequency chart comprising, for:
a plurality of groups of model configurations of the stratified target environment, each comprising at least two model configurations of the stratified target environment differing from one another only by the parameter of interest,
and a plurality of irradiation case groups, each comprising at least two irradiation cases differing from one another only in a modulation frequency of a light source, each irradiation case comprising a set of irradiation parameter values,
a plurality of nodal multiplets, each nodal multiplet comprising characteristics common to all the elements of a given group of model configurations of the stratified target environment and a plurality of associated nodal modulation frequencies, for which the acoustic or thermal signal detected by the detection cell in response to irradiation by the light source exhibits a correlation below a predetermined threshold with the parameter of interest;
and an adaptation module-comprising a processor and exchanging information with the detection cell and the light source irradiation parameter control device;
b) the adaptation module selects a model configuration of the initial stratified target environment for irradiation;
c) the processor of the adaptation module:
determines in the nodal frequency chart a plurality of nodal modulation frequencies associated with a group of model configurations of the stratified target environment to which the chosen model configuration of the stratified environment belongs,
then determines at least one particular modulation frequency on the basis of said plurality of nodal modulation frequencies, said particular modulation frequency being a weighted average of at least two of said plurality of nodal modulation frequencies and being bounded by two nodal modulation frequencies,
and determines a particular irradiation case for the chosen model configuration of the stratified target environment, the particular irradiation case comprising the at least one particular modulation frequency;
d) the light source irradiates the stratified target environment according to the set of irradiation parameters of said particular irradiation case;
e) the detection cell detects an acoustic or thermal signal generated in response to irradiation;
f) the processor of the adaptation module determines the parameter of interest on the basis of the acoustic or thermal signal detected.

2. Measurement method according to claim 1 wherein:
the processor of the adaptation module further implements an inverse modeling algorithm receiving as input an irradiation case and an acoustic or thermal signal and providing as output a model configuration of the stratified target environment and a value of the parameter of interest,
step f) comprises the following steps:
f1) the adaptation module processor receives as input the acoustic or thermal signal detected by the detection cell and the particular irradiation case used for irradiation, and returns as output from the inverse modeling algorithm a model configuration of the current stratified target environment and an estimated value of the parameter of interest;
f2) the adaptation module processor evaluates the chosen stratified target environment model configuration by comparison with the current stratified target environment model configuration, and, only if CMirrad is different from CMmes: g) the adaptation module receives as input the model configuration of the current stratified target environment and returns as output a new model configuration of the chosen stratified target environment for irradiation, then c), d), e) and f are repeated;
f3) the value of the parameter of interest measured by the sensor is the last value of the parameter of interest estimated.

3. Measurement method according to claim 1, g) further comprising repeating f2) at the end of f1).

4. Measurement method according to claim 1 comprising beforehand:
I—by means of a processor and a database of model configurations comprising multiplets (model configuration of the stratified target environment, irradiation case, parameter of interest) and an acoustic or thermal signal detected by the detection cell associated with each of said multiplets, a nodal frequency chart is generated for:
a plurality of groups of model configurations,
and a plurality of groups of irradiation cases,
the nodal frequency chart comprises a plurality of nodal multiplets, each nodal multiplet comprising characteristics common to all the elements of a group of model configurations of the stratified target environment and a plurality of associated nodal modulation frequencies, and this nodal frequency abacus is stored in the memory of the non-invasive sensor.

5. Measurement method according to claim 4 comprising:
II—a processor learns at least one inverse modeling algorithm from the model configuration database, and the at least one inverse modeling algorithm is stored in the memory of the non-invasive sensor.

6. Measurement method according to claim 4 in which at least one of the acoustic or thermal signals detected by the detection cell associated with the multiplets stored in the model configuration database are simulated, i.e. generated by means of a computerized simulation device.

7. Measurement method according to claim 1 wherein said particular irradiation case for the chosen model configuration determined by the adaptation module in step c) further comprises at least a nodal modulation frequency.

8. A non-invasive sensor based on photoacoustic or photothermal detection configured to measure a parameter of interest in a stratified target environment comprising:
a light source,
a device for controlling the irradiation parameters of the light source,
a detection cell configured to detect an acoustic or thermal signal,
a memory in which is stored a nodal frequency abacus comprising, for:
a plurality of groups of model configurations of the stratified target environment, each comprising at least two model configurations of the stratified target environment differing from one another only by the parameter of interest,
and a plurality of groups of irradiation cases, each comprising at least two irradiation cases differing from one another only in a modulation frequency of a light source, each irradiation case comprising a set of irradiation parameter values, a plurality of nodel multiplets, each nodal multiplet comprising characteristics common to all the elements of a given group of model configurations of the stratified target environment and a plurality of associated nodal modulation frequencies, for which the acoustic or thermal signal detected by the detection cell in response to irradiation by the light source exhibits a correlation below a predetermined threshold with the parameter of interest,
the non-invasive sensor further comprising an adaptation module comprising a processor and adapted to exchange information with the detection cell and the light source irradiation parameter control device, the adaptation module being further configured to:
select a model configuration of the initial stratified target environment,
determine in the nodal frequency chart a plurality of nodal modulation frequencies associated with a group of model configurations to which the selected stratified target environment model configuration belongs,
determine at least one particular modulation frequency on the basis of said plurality of nodal modulation frequencies, said particular modulation frequency being a weighted average of at least two of said plurality of nodal modulation frequencies and being bounded by two nodal modulation frequencies;
determine a particular irradiation case for the selected irradiation model configuration comprising the at least one particular modulation frequency;
transmit a particular irradiation case to the light source irradiation parameter control device,
receive an acoustic or thermal signal detected by the detection cell,
determine the value of the parameter of interest on the basis of the acoustic or thermal signal detected.

9. A non-invasive sensor according to claim 8 wherein the processor of the adaptation module is further adapted to:
implement an inverse modeling algorithm receiving as input an irradiation case comprising a set of irradiation parameters and an acoustic or thermal signal and providing as output a model configuration of the stratified target environment and a value of the parameter of interest;
using the inverse modeling algorithm, determine a current model configuration of the stratified target environment and an estimated value of the parameter of interest on the basis of a detected photoacoustic or photothermal signal received and a selected irradiation case;
evaluate a chosen model irradiation configuration of the stratified target environment by comparison with a current model configuration of the stratified target environment;
only if the model irradiation configuration of the chosen target stratified environment and the model configuration of the current target stratified environment compared are different: determine a new model irradiation configuration of the selected stratified target environment when it receives a model irradiation configuration of the current stratified target environment,
determine in the nodal frequencies chart a plurality of nodal modulation frequencies associated with a group of model configuration of the target stratified environment to which the new model configuration of the chosen stratified environment belongs,
then determine at least one particular modulation frequency on the basis of said plurality of nodal modulation frequencies, said particular modulation frequency being a weighted average of at least two of said plurality of nodal modulation frequencies and being delimited by two nodal modulation frequencies,
determine a new particular irradiation case for the new model configuration of the chosen target stratified environment, the particular irradiation case comprising the at least one particular modulation frequency,
and transmit the new particular irradiation case to the device for controlling the irradiation parameters of the light source so that the light source irradiates the target stratified environment following the set of irradiation parameters of said new particular irradiation case, that the detection cell detects an acoustic or thermal signal generated in response to this new irradiation and the processor of the adaptation module determines the parameter of interest based on the new acoustic or thermal signal detected;
determine the measured parameter of interest value on the basis of the last estimated value of the parameter of interest.

10. Non-invasive sensor according to claim 8 in which at least two of the devices among the light source, the device for controlling the irradiation parameters of the light source, the detection cell configured to detect an acoustic or thermal signal, the memory in which an abacus of nodal frequencies is stored, and the adaptation module are mechanically independent of each other.

11. A computer program comprising instructions which lead the non-invasive sensor according to claim 8 to perform the steps of the method of measuring a parameter of interest in a stratified target environment by means of a non invasive sensor based on photoacoustic detection or photothermal detection, wherein the method comprises:
a) a sensor is provided comprising:
a light source,
a device for controlling a plurality of irradiation parameters of the light source, the plurality of irradiation parameters comprising at least one frequency for modulating the intensity of the light source,
a detection cell configured to detect an acoustic or thermal signal,
a memory in which is stored a nodal frequency abacus comprising, for:
a plurality of groups of model configurations of the stratified target environment, each comprising at least two model configurations of the stratified target environment differing from one another only by the parameter of interest,
and a plurality of irradiation case groups, each comprising at least two irradiation cases differing from one another only in a modulation frequency of a light source, each irradiation case comprising a set of irradiation parameter values,
a plurality of nodal multiplets, each nodal multiplet comprising characteristics common to all the elements of a given group of model configurations of the stratified target environment and a plurality of associated nodal modulation frequencies, for which the acoustic or thermal signal detected by the detection cell in response to irradiation by the light source exhibits a correlation below a predetermined threshold with the parameter of interest;
and an adaptation module comprising a processor and exchanging information with the detection cell and the light source irradiation parameter control device;
b) the adaptation module selects a model configuration of the initial stratified target environment for irradiation;
c) the processor of the adaptation module:
determines in the nodal frequency abacus a plurality of nodal modulation frequencies associated with a group of model configurations of the stratified target environment to which the chosen model configuration of the stratified environment belongs,
then determines at least one particular modulation frequency on the basis of said plurality of nodal modulation frequencies, said particular modulation frequency being a weighted average of at least two of said plurality of nodal modulation frequencies and being bounded by two nodal modulation frequencies,
and determines a particular irradiation case for the chosen model configuration of the stratified target environment, the particular irradiation case comprising the at least one particular modulation frequency;
d) the light source irradiates the stratified target environment according to the set of irradiation parameters of said particular irradiation case;
e) the detection cell detects an acoustic or thermal signal generated in response to irradiation;
f) the processor of the adaptation module determines the parameter of interest on the basis of the acoustic or thermal signal detected.
